(19) United States
(12) Patent Application Publication
(10) Pub. No.: US 2026/0198854 A1
(43) Pub. Date: Jul. 16, 2026

(54) INTELLIGENT WEARABLE DEVICE

(71) Applicant: Huzhou Luxshare Precision Industry Co., Ltd., Huzhou City (CN)

(72) Inventors: Jingren CAO, Huzhou (CN); Xin TIAN, Huzhou (CN)

(73) Assignee: Huzhou Luxshare Precision Industry Co., Ltd., Huzhou City (CN)

(21) Appl. No.: 19/089,664

(22) Filed: Mar. 25, 2025

(30) Foreign Application Priority Data
Jan. 15, 2025 (CN) 202520101294.0

(51) Int. Cl. A61B 5/00; A44C 9/00; A61B 5/0205

(52) U.S. Cl. CPC A61B 5/6802; A44C 9/0053; A61B 5/02055

(57) ABSTRACT

An intelligent wearable device includes: an inner frame; a flexible belt provided on an outer periphery of the inner frame; an adjusting assembly circumferentially provided on the inner frame and located on a side of the flexible belt, the adjusting assembly abutting against the flexible belt; and a driving assembly provided on an outer side of the adjusting assembly and connected to the adjusting assembly. When the driving assembly rotates in a first direction, the adjusting assembly is driven to rotate in a first circumferential direction of the inner frame, so as to press the flexible belt to be deformed to reduce an inner diameter of the inner frame. When the driving assembly rotates in a second direction, the adjusting assembly is driven to rotate in a second circumferential direction of the inner frame, the flexible belt is elastically reset to increase the inner diameter of the inner frame.

### CROSS-REFERENCE TO RELATED APPLICATIONS

This application claims priority to Chinese patent application No. 2025201012940, entitled “INTELLIGENT WEARABLE DEVICE”, filed on Jan. 15, 2025, the disclosure of which is hereby incorporated by reference in its entirety.

### TECHNICAL FIELD

The present disclosure pertains to the technical field of wearable devices, and relates to an intelligent wearable device.

### BACKGROUND

As an innovative product in the field of intelligent wearable devices, smart rings have received wide attention in recent years for their convenience and practicality. Such devices can monitor and feedback human health indicators in real time by integrating various technical means.

However, there are obvious shortcomings in the size adjustment of smart rings on the existing market. On the one hand, some smart rings have a fixed size design that matches the fingers of different users by providing different inner diameters. Such design not only increases production costs because of the need to produce multiple sizes to meet market demands, but also makes it difficult to ensure the best comfort for every user. Due to the difference in finger size, fixed-size rings often do not fit all users'fingers perfectly, which affects comfort of wearing and accuracy of health monitoring. On the other hand, in order to solve the problem of size adaptation, some smart rings are designed as two parts that can move relative to each other. The two parts are tightened together by a spring structure, so as to fit tightly around the finger of the user. However, the aesthetic of the smart ring is greatly reduced, and the tightness of the smart ring cannot be adjusted freely according to different sizes of the finger. In addition, long-term use of the spring structure may result in a weakening of elasticity of the spring structure, thereby affecting the stability and comfort of wearing.

In view of the above, such the existing intelligent wearable devices as smart rings have problems, such as insufficient size adaptation, inflexible size adjustment and poor comfort.

### SUMMARY

Therefore, it is necessary to provide an intelligent wearable device to address the problems of insufficient size adaptation and inflexible size adjustment of the existing wearable devices.

The intelligent wearable device includes:

an inner frame;

a flexible belt provided on an outer periphery of the inner frame;

an adjusting assembly circumferentially provided on the inner frame and located on a side of the flexible belt away from the inner frame, wherein the adjusting assembly abuts against the flexible belt; and

a driving assembly provided on an outer side of the adjusting assembly and connected to the adjusting assembly;

the driving assembly is capable of rotating in a first direction, when the driving assembly rotates in the first direction, the adjusting assembly is driven to rotate in a first circumferential direction of the inner frame, so as to press the flexible belt to be deformed to reduce an inner diameter of the inner frame; or

the driving assembly is capable of rotating in a second direction, when the driving assembly rotates in the second direction, the adjusting assembly is driven to rotate in a second circumferential direction of the inner frame, the flexible belt is elastically reset to increase the inner diameter of the inner frame.

In one or more embodiment, the adjusting assembly includes at least one rotating member in transmission connection with the driving assembly.

In one or more embodiment, the rotating member includes a cam and a camshaft, the cam is longitudinally provided on the outer periphery of the inner frame and is located on the side of the flexible belt away from the inner frame, and the camshaft longitudinally extends through an end of the cam; a distance between a first portion of a side surface of the cam and the camshaft is a first radius, a distance between a second portion of the side surface of the cam and the camshaft is a second radius, and the first radius is larger than the second radius.

In one or more embodiment, the cam has a cylindrical structure, and outer peripheries of portions of the camshaft extending out of an upper end of the cam and a lower end of the cam are provided with tooth structures, respectively.

In one or more embodiment, a plurality of rotating members are provided, and a plurality of cams are evenly spaced apart on the outer periphery of the inner frame; and wherein a portion extending outward from a top portion of the inner frame and a portion extending outward from a bottom portion of the inner frame are respectively provided with a plurality of through holes that are evenly spaced apart, and upper end and lower end of each camshaft are respectively inserted in a pair of the through holes respective located at the top portion and bottom portion of the inner frame.

In one or more embodiment, the driving assembly is a middle frame having an annular structure, and an inner circumference of the middle frame is provided with inner tooth surfaces respectively engaged with outer tooth surfaces provided on an outer periphery of an upper end and an outer periphery of a lower end of the camshaft.

In one or more embodiment, the intelligent wearable device further includes a damping member, the damping member is provided at a bottom portion of the outer periphery of the inner frame, and a surface of the damping member is provided with a tooth surface engaged with the inner tooth surface.

In one or more embodiment, the damping member is a protrusion extending outward from an outer wall of the inner frame.

In one or more embodiment, the second portion of the side surface of the cam is opposite to the first portion of a side surface of the cam.

In one or more embodiment, the rotating member includes an eccentric rotation shaft, a first radial portion, and a second radial portion, the first radial portion is a portion of the edge of the cam is farthest from the rotation shaft, the second radial portion is a portion of the edge of the cam is closest to the rotation shaft, the first radius is a distance from the first radial portion of the cam to the rotation shaft, and the second radius is a distance from the second radial portion of the cam to the rotation shaft.

In one or more embodiment, a surface of the cam is a curved surface with a smooth transition.

In one or more embodiment, the inner frame is flexible.

In one or more embodiment, the intelligent wearable device further includes an upper cover and a lower cover, a portion extending outward from a top portion of the inner frame and a portion extending outward from a bottom portion of the inner frame are respectively provided with a plurality of fixing holes that are evenly spaced apart, a side of the upper cover adjacent to the top portion of the inner frame and a side of the lower cover adjacent to the bottom portion of the inner frame are respectively provided with a plurality of fixing posts, the plurality of fixing posts of the upper cover are in one-to-one correspondence with the plurality of fixing posts of the lower cover, each fixing post of the upper cover and corresponding fixing post of the lower cover respectively extend through the corresponding fixing holes and are located at a same side of one of a plurality of bending positions of the flexible belt.

In one or more embodiment, the intelligent wearable device further includes:

a plurality of detection sensors provided on a side of the flexible belt away from the adjusting assembly, the plurality of detection sensors being configured to monitor health condition of a user; and

a power supply provided on the outer peripheral of the inner frame and electrically connected to the flexible belt.

In an embodiment, the plurality of detection sensors include at least two of a heart rate sensor, a blood oxygen sensor and a temperature sensor.

In an embodiment, the first circumferential direction is the same as the first direction.

In an embodiment, the second circumferential direction is the same as the second direction and opposite to the first direction.

In an embodiment, the flexible belt is a closed loop structure.

In an embodiment, a contact portion between the flexible belt and the adjusting assembly is provided with a cushioning material.

In an embodiment, the cushioning portion is a sponge or a foam.

When the above-mentioned intelligent wearable device is in use, the inner diameter of the intelligent wearable device can be adjusted quickly by rotating the driving assembly without removing any component, so that the intelligent wearable device is suitable for users with different finger sizes and meets the wearing needs of different people, thereby improving the applicability and convenience.

### BRIEF DESCRIPTION OF THE DRAWINGS

FIG. 1 is an exploded view of an intelligent wearable device.

FIG. 2 is a partial perspective view of the intelligent wearable device in a first state according to an embodiment of the present disclosure.

FIG. 3 is a top view of an adjusting assembly and a flexible belt in the first state according to an embodiment of the present disclosure.

FIG. 4 is a partial perspective view of the intelligent wearable device in the second state.

FIG. 5 is a top view of an adjusting assembly and a flexible belt in a second state according to an embodiment of the present disclosure.

FIG. 6 is a perspective view of a rotating member according to an embodiment of the present disclosure.

FIG. 7 is a perspective view of an inner frame according to an embodiment of the present disclosure.

FIG. 8 is a perspective view of a driving assembly according to an embodiment of the present disclosure.

FIG. 9 is a perspective view of a flexible belt according to an embodiment of the present disclosure.

Reference Numerals: 1. inner frame; 11. through hole; 12. fixing hole; 2. flexible belt; 21. detection sensor; 22. bending portion; 3. adjusting assembly; 30. rotating member; 31. rotation shaft; 32. first radial portion; 33. second radial portion; 301. cam; 302. camshaft; 303. outer tooth surface; 4. driving assembly; 40. middle frame; 41. inner tooth surface; 5. damping member; 50. protrusion; 51. tooth surface; 6. upper cover; 61. fixing post; 7. lower cover; 8. power supply.

### DETAILED DESCRIPTION

In order to make the objectives, features, and advantages of the present disclosure more apparent and understandable, the specific implementations of the present disclosure will be explained in detail below in conjunction with the accompanying drawings. In the following description, numerous specific details are set forth in order to facilitate a thorough understanding of the present disclosure. However, the present disclosure can be implemented in many other ways different from those described herein, and those skilled in the art can make similar modifications without departing from the spirit of the application, so that the present disclosure is not limited to the specific embodiments disclosed below.

In the description of the present disclosure, it is to be understood that the terms “center”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counterclockwise”, “axial”, “radial”, “circumferential”, etc., if being used herein, indicate the orientations or positional relationships on the basis of the drawings. These terms are only intended for facilitating illustrating the present disclosure and simplifying the illustration, rather than indicating or implying that the devices or elements referred thereto have to present particular orientations, and be constructed and operated in particular orientations, and therefore cannot be construed as limiting the present disclosure.

In addition, the terms “first” and “second”, if being used herein, are only intended for illustrative purposes, rather than being construed as indicating or implying relative importance or implicitly designating the number of the technical features as indicated. Thus, the features modified by “first” and “second” may explicitly or implicitly include at least one said feature. In the description of the present disclosure, the term “a plurality of”, if being used herein, means at least two, for example two or three, unless otherwise explicitly and specifically defined.

In the present disclosure, unless otherwise expressly specified and defined, the terms “mounted”, “connected to”, “coupled” and “fixed”, if being used herein, should be understood in a broad sense. For example, they can mean fixedly connected or detachably connected, or integrated; mechanically connected or electrically connected; directly connected or indirectly connected through an intermediate medium, or in an interior communication or mutual interaction relationship between two elements, unless otherwise specifically defined. For those of ordinary skill in the art, the specific meanings of the above-described terms in the present disclosure may be understood according to specific circumstances.

In the present disclosure, unless otherwise expressly stated and defined, the first feature, if being referred to as being located “above” or “below” the second feature, may be in direct contact with the second feature, or in indirect contact with the second feature via an intermediate feature. Moreover, the first feature, when being referred to as being provided “on”, “above” and “over” the second feature, may be provided right above or obliquely above the second feature, or simply provided at a level higher than the second feature. The first feature, when being referred to as being provided “under”, “below” and “beneath” the second feature, may be provided directly below or obliquely below the second feature, or simply provided at a level lower than the second feature.

It should be noted that, if an element is referred to as being “fixed to” or “provided on” another element, it may be directly on the other element or may also be present with an intermediate element. If an element is referred to as being “connected” to another element, it may be directly connected to the other element or may be present with an intermediate element at the same time. The terms “vertical”, “horizontal”, “up”, “down”, “left”, “right” and similar expressions, if being used herein, are only for the purpose of illustration, rather than presenting the only ways for implementation.

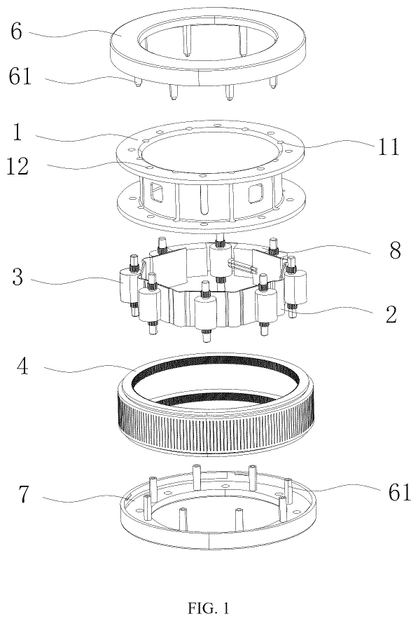

Referring to FIGS. 1 to 5, FIGS. 1 to 5 respectively show an exploded view and partial perspective views of an intelligent wearable device and top views of an adjusting assembly 3 and a flexible belt 2 according to an embodiment of the present disclosure.

Referring to FIG. 1, an embodiment of the present disclosure provides an intelligent wearable device, including an inner frame 1, a flexible belt 2, an adjusting assembly 3, and a driving assembly 4. The intelligent wearable device provided by the embodiment of the present disclosure takes a smart ring as an example, and a function for adjusting an inner diameter of the smart ring is achieved by integrating the above components in the smart ring, so as to adapt to the finger sizes of different users.

Specifically, the flexible belt 2 is provided on the outer periphery of the inner frame 1. The adjusting assembly 3 is circumferentially provided on the inner frame 1 and is located on a side of the flexible belt 2 away from the inner frame 1, and the adjusting assembly 3 abuts against the flexible belt 2. The driving assembly 4 is provided on an outer side of the adjusting assembly 3 and is connected to the adjusting assembly 3. When the driving assembly 4 rotates in a first direction, the adjusting assembly 3 is driven to rotate in a first circumferential direction of the inner frame 1 to push the flexible belt 2 to be deformed, so that the flexible belt 2 presses a flexible portion of the inner frame 1, thereby reducing the inner diameter of the inner frame 1. When the driving assembly 4 rotates in a second direction, the adjusting assembly 3 is driven to rotate in a second circumferential direction of the inner frame 1, so that the pressing of the adjusting assembly 3 on the flexible belt 2 and the flexible portion of the inner frame 1 is released, and the flexible belt 2 and the flexible portion of the inner frame 1 are elastically reset to increase the inner diameter of the inner frame 1.

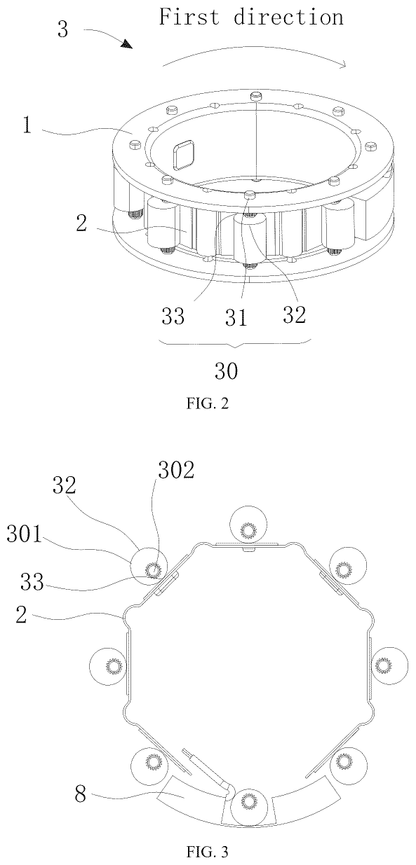

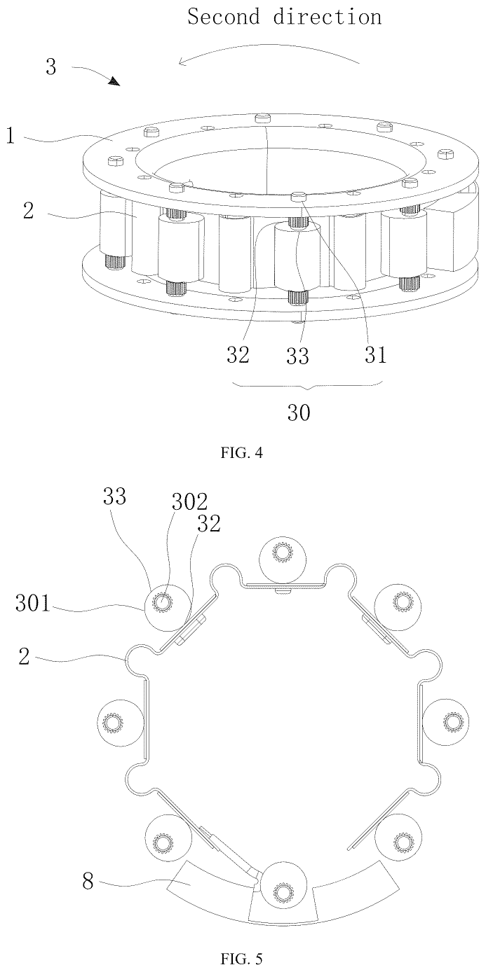

Referring to FIGS. 2 and 4, in use, when a user needs to adjust an inner diameter of an intelligent wearable device (e.g., a smart ring), the user only needs to rotate the driving assembly 4 in the first direction, that is, clockwise or counterclockwise, to drive the adjusting assembly 3 to rotate in the first circumferential direction of the inner frame 1. The adjusting assembly 3 pushes the flexible belt 2 to be deformed, and the flexible belt 2 presses the flexible portion of the inner frame 1, so that the inner frame 1 is deformed inward, thereby gradually reducing the effective inner diameter of the inner frame 1.

In the present embodiment, the first circumferential direction of the inner frame 1 is the same as the first direction of the rotation of the driving assembly 4, which may be clockwise or counterclockwise, depending on usage requirements.

Correspondingly, the driving assembly 4 is rotated in the second direction to drive the adjusting assembly 3 to rotate in the second circumferential direction of the inner frame 1, so that the pressing of the adjusting assembly 3 on the flexible belt 2 and the inner frame 1 is released. The flexible belt 2 and the inner frame 1 are elastically reset, so that the effective inner diameter of the inner frame 1 is gradually increased, and the process of adjusting the inner diameter of the intelligent wearable device is achieved. In the present embodiment, the second circumferential direction of the inner frame 1 is the same as the second direction of the rotation of the driving assembly 4 and opposite to the first direction of the rotation of the driving assembly 4, which may be counterclockwise or clockwise, or may be understood as a direction in which the flexible belt 2 is elastically reset, depending on usage requirements.

In an embodiment, the inner frame 1 serves as the main structure of the intelligent wearable device and is made of a flexible material, such as silica gel or thermoplastic polyurethane (TPU), so that the inner frame 1 is flexible. This material is not only lightweight but also has good elasticity, which can adapt to the bending and deformation of fingers.

Referring to FIG. 9, FIG. 9 is a perspective view of the flexible belt 2 according to an embodiment of the present disclosure. In some embodiments, the flexible belt 2 adopts a flexible printed circuit (FPC) technology. The intelligent wearable device further includes a plurality of detection sensors 21, such as a heart rate sensor, a blood oxygen sensor, a temperature sensor, and the like. The detection sensors 21 are provided on a side of the flexible belt 2 away from the adjusting assembly 3, so as to monitor the health condition of the user. The flexible belt 2 is provided on the outer periphery of the inner frame 1 to form a closed loop structure for fitting the fingers of the user.

Further, a contact portion between the flexible belt 2 and the adjusting assembly 3 is provided with a cushioning portion, such as a sponge, a foam, etc., to reduce wear, improve comfort of wearing, and help to prolong the service life.

In an embodiment, the intelligent wearable device further includes a power supply 8 provided on the outer periphery of the inner frame 1 and electrically connected to the flexible belt 2, so as to provide power for components such as the detection sensors 21.

In an embodiment, the adjusting assembly 3 is provided on the inner frame 1 along the circumferential direction of the inner frame 1 and is located on the side of the flexible belt 2 away from the inner frame 1. When the adjusting assembly 3 is rotated, the flexible belt 2 can be pushed to deform or reset by the elasticity of the flexible belt 2, thereby changing the effective inner diameter of the inner frame 1.

In an embodiment, the driving assembly 4 is provided on the outer side the adjusting assembly 3 and is connected to the adjusting assembly 3. The driving assembly 4 may be a knob-type middle frame 40 for ease of user operation.

As described above, when the above-mentioned intelligent wearable device is in use, the inner diameter of the intelligent wearable device can be adjusted quickly only by rotating the driving assembly 4 without removing or replacing any component, so that the intelligent wearable device is suitable for users with different finger sizes and meets the wearing needs of different people, thereby improving the applicability and convenience. In addition, the design of the soft inner frame 1 and the flexible belt 2 enables the intelligent wearable device to be closely fit the user's finger, thereby improving the comfort of wearing.

In an embodiment, the adjusting assembly 3 includes at least one rotating member 30 in transmission connection with the driving assembly 4. By rotating the driving assembly 4, a rotational force is transmitted to the adjusting assembly 3 to provide a driving force for the rotation of the adjusting assembly 3.

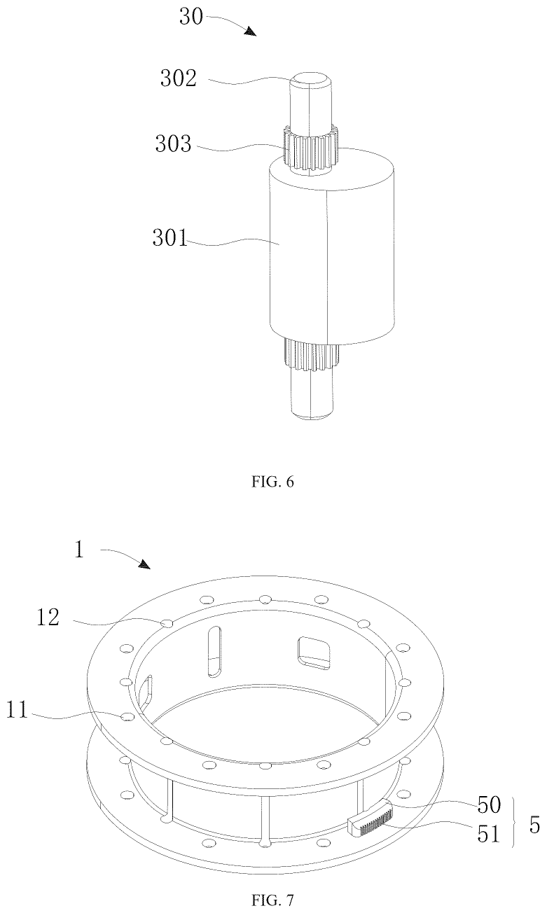

Referring to FIG. 6, FIG. 6 is a perspective view of the rotating member 30 according to an embodiment of the present disclosure. In some embodiments, the rotating member 30 includes a cam 301 and a camshaft 302. The tension of the flexible belt 2 is precisely adjusted through the rotation of the cam 301, so as to improve the wearing experience and ease of operation.

In an embodiment, the cam 301 is longitudinally provided on the outer periphery of the inner frame 1 and is located on the side of the flexible belt 2 away from the inner frame 1, and the camshaft 302 longitudinally extends through an end of the cam 301.

Specifically, the cam 301 is longitudinally provided on the outer periphery of the inner frame 1 and is located on the side of the flexible belt 2 away from the inner frame 1. The cam 301 is designed to be asymmetric in shape and its contour line varies in a circumferential direction thereof to achieve different radial distances.

Further, the camshaft 302 longitudinally extends through an end of the cam 301 and serves as a central shaft of rotation of the cam 301, so as to ensure that the cam 301 can rotate stably and smoothly.

In an embodiment, a distance between a first portion of a side surface of the cam 301 and the camshaft 302 is a first radius, a distance between a second portion of the side surface of the cam 301 and the camshaft 302 is a second radius, and the first radius is larger than the second radius. The second portion of the side surface of the cam 301 is opposite to the first portion of a side surface of the cam 301.

In use, when a user needs to adjust the wearing tightness of the intelligent wearable device, the tension state of the flexible belt 2 can be changed by rotating the cam 301. Since the first radius is larger than the second radius, when the cam 301 rotates until the first portion of the side surface of the cam 301 is pressed against the flexible belt 2, the flexible belt 2 is stretched more. While when the cam 301 rotates until the second portion of the side of the cam 301 presses against the flexible belt 2, the flexible belt 2 is relatively loose.

Referring to FIGS. 3 to 5, FIG. 3 is a top view of the adjusting assembly 3 and the flexible belt 2 in a first state according to an embodiment of the present disclosure, FIG. 4 is a partial perspective view of the intelligent wearable device in a second state, and FIG. 5 is a top view of the adjusting assembly 3 and the flexible belt 2 in the second state according to an embodiment of the present disclosure. In some embodiments, the rotating member 30 includes an eccentric rotation shaft 31, a first radial portion 32, and a second radial portion 33.

Specifically, the first radial portion 32 is a portion of the edge of the cam 301 is farthest from the rotation shaft 31. The second radial portion 33 is a portion of the edge of the cam 301 is closest to the rotation shaft 31. The first radius is the distance from the first radial portion 32 of the cam 301 to the rotation shaft 31, and the second radius is the distance from the second radial portion 33 of the cam 301 to the rotation shaft 31. The first radial portion 32 and the second radial portion 33 are eccentrically provided, that is, the first radius is larger than the second radius. In this way, during the rotation of the cam 301, the flexible belt 2 will be subject to unevenly varying pushing force, so as to realize the dynamic adjustment of the tension of the flexible belt 2 and the adjustment of the inner diameter.

In an embodiment, the inner diameter of the inner frame 1 gradually decreases during the rotation of the rotating member 30 from a state where the second radial portion 33 abuts against the flexible belt 2 to a state where the first radial portion 32 abuts against the flexible belt 2.

Specifically, since the rotating member 30 of an eccentric design, when the driving assembly 4 rotates in the first direction, the rotating member 30 rotates from a state where the second radial portion 33 (i.e., the end that is close to the center of the inner frame 1) abuts against the flexible belt 2 to a state where the first radial portion 32 (i.e., the end that is far away from the center of the inner frame 1) abuts against the flexible belt 2, and the rotating member 30 gradually presses the flexible belt 2 and the inner frame 1, so as to increase the deformation of the inner frame 1, thereby reducing the inner diameter of the inner frame 1.

Correspondingly, when the driving assembly 4 rotates in an opposite direction, the rotating member 30 rotate reversely in the second direction, that is, the rotating member 30 rotates from a state where the first radial portion 32 abuts against the flexible belt 2 to a state where the second radial portion 33 abuts against the flexible belt 2, so that the pressing of the rotating member 30 on the flexible belt 2 and the inner frame 1 is released. The flexible belt 2 and the flexible portion of the inner frame 1 are reset due to the elasticity, and the inner diameter of the inner frame 1 increases accordingly.

As described above, by providing the rotating member 30 having the cam 301 and the camshaft 302, not only the flexible adjustment of the wearing tightness is realized, but also the convenience and comfort of the user's operation are improved. Moreover, through the design of the adjusting assembly 3, the inner diameter of the intelligent wearable device can be precisely and conveniently adjusted to meet the use requirements of different users.

In an embodiment, the cam 301 has a cylindrical structure, and outer peripheries of portions of the camshaft 302 extending out of the upper and lower ends of the cam 301 are provided with tooth structures, respectively.

Specifically, the cam 301 having the cylindrical structure is provided on the outer periphery of the inner frame 1, and the tension of the flexible belt 2 is adjusted by the rotation of the cam 301. The surface of the cam 301 is a curved surface with a smooth transition to ensure smoothness and stability during rotation.

Further, the outer peripheries of the portions of the camshaft 302 extending out of the upper and lower ends of the cam 301 are provided with outer tooth surfaces 303, respectively. The outer tooth surfaces 303 are configured to be engaged with inner tooth surfaces 41 of the driving assembly 4, so that the cam 301 can rotate together with the camshaft 302, thereby achieving accurate adjustment of the tension of the flexible belt 2.

Referring to FIG. 7, FIG. 7 is a perspective view of an inner frame 1 according to an embodiment of the present disclosure. In some embodiments, a plurality of rotating members 30 are provided, and a plurality of cams 301 are evenly spaced apart on the outer periphery of the inner frame 1.

Specifically, the plurality of cams 301 are evenly spaced apart on the outer periphery of the inner frame 1. Each cam 301 is connected to the inner frame 1 through the camshaft 302. A certain interval is maintained between the cams 301, so as to ensure stability and accuracy of adjustment during rotation, and realize uniform adjustment of the inner diameter of the intelligent wearable device.

Further, a portion extending outward from a top portion of the inner frame 1 and a portion extending outward from a bottom portion of the inner frame 1 are respectively provided with a plurality of through holes 11 that are evenly spaced apart. The upper and lower ends of each camshaft 302 are respectively inserted in a pair of through holes 11 respectively located at the top portion and bottom portion of the inner frame 1 to connect the cam 301 and the inner frame 1, so as to ensure that the camshaft 302 can be stably mounted on the inner frame 1, and allow the cam 301 to rotate about the axial direction of the camshaft 302. Through such an arrangement, not only the stability of the structure is further improved, but also the adjustment of the inner diameter of the intelligent wearable device is more flexible and accurate.

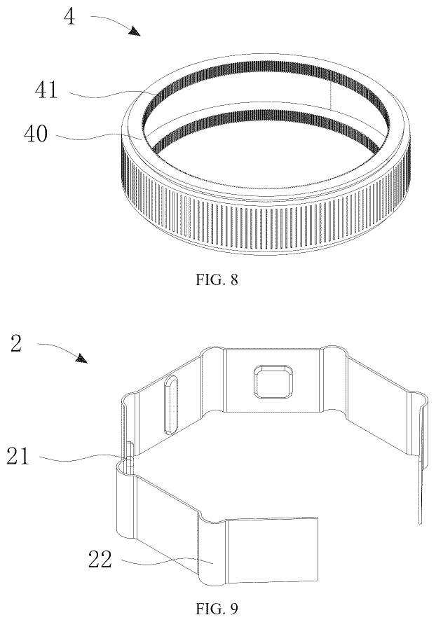

Referring to FIG. 8, FIG. 8 is a perspective view of a driving assembly 4 according to an embodiment of the present disclosure. In some embodiments, the driving assembly 4 is a middle frame 40 having an annular structure. An inner circumference of the middle frame 40 is provided with inner tooth surfaces 41 respectively engaged with the outer tooth surfaces 303 provided on the outer periphery of the upper end and the outer periphery of the lower end of the camshaft 302.

Specifically, the middle frame 40 having the annular structure is provided on the periphery of the cam 301 having the cylindrical structure. The inner circumference of the middle frame 40 is provided with inner tooth surfaces 41 respectively engaged with the outer tooth surfaces 303 provided on the outer periphery of the upper end and the outer periphery of the lower end of the camshaft 302. The outer tooth surfaces 303 are engaged with the inner tooth surfaces 41, the cams 301 provided eccentrically can be driven to rotate together about the axial direction of the camshaft 302, thereby changing the tension state of the flexible belt 2, and adjusting the inner diameter of the inner frame 1.

As described above, the plurality of cams 301 are spaced apart on the outer periphery of the inner frame 1, so that the overall structure of the intelligent wearable device is compact and aesthetically pleasing. By rotating the cam 301, the inner diameter of the intelligent wearable device can be adjusted flexibly to meet the needs of different users. In addition, the surface of the cam 301 is a curved surface with a smooth transition, which ensures smoothness and comfort during rotation.

Referring to FIG. 7, FIG. 7 is a perspective view of an inner frame 1 according to an embodiment of the present disclosure. In some embodiments, the intelligent wearable device further includes a damping member 5 provided at a bottom portion of the outer periphery of the inner frame 1. A surface of the damping member 5 is provided with a tooth surface 51 engaged with the inner tooth surface 41.

Specifically, the damping member 5 is provided at the bottom portion of the outer periphery of the inner frame 1, and the damping member 5 is a protrusion 50 extending outward from an outer wall of the inner frame 1, which enhances the structural strength of the inner frame 1.

Further, the tooth surface 51 provided on the surface of the protrusion 50 is engaged with the inner tooth surface 41 provided on the inner periphery of the middle frame 40, which enables the cam 301 to be restricted by the damping member 5 during rotation, thereby avoiding looseness or instability caused by excessive rotation. Meanwhile, by adjusting the engaging degree of the tooth surface 51, the rotational resistance of the cam 301 can also be adjusted, so as to meet the preference of different users for the tactile sensation of adjusting.

As described above, by providing the damping member 5 engaged with the middle frame 40, accurate adjustment of the inner diameter of the intelligent wearable device is achieved. Meanwhile, looseness or instability caused by excessive rotation is effectively avoided, thereby ensuring stability during wearing. By adjusting the engaging degree of the tooth surface 51, the rotational resistance of the cam 301 can be adjusted, so that the inner diameter of the inner frame 1 can be locked, and at the same time, the preference of different users for the tactile sensation of adjusting can be satisfied.

Referring to FIG. 1, FIG. 1 is an exploded view of the intelligent wearable device according to an embodiment of the present disclosure. In some embodiments, the intelligent wearable device further includes an upper cover 6 and a lower cover 7, which are provided at the top portion and bottom portion of the inner frame 1, respectively and are configured to fix and protect internal components.

In an embodiment, the portion extending outward from the top portion of the inner frame 1 and the portion extending outward from the bottom portion of the inner frame 1 are respectively provided with a plurality of fixing holes 12 that are evenly spaced apart. A side of the upper cover 6 adjacent to the top portion of the inner frame 1 and a side of the lower cover 7 adjacent to the bottom portion of the inner frame 1 are respectively provided with a plurality of fixing posts 61. The plurality of fixing posts 61 of the upper cover 6 are in one-to-one correspondence with the plurality of fixing posts 61 of the lower cover 7, each fixing post 61 of the upper cover 6 and corresponding fixing post 61 of the lower cover 7 respectively extend through the corresponding fixing holes 12 and are located at a same side of one of a plurality of bending positions of the flexible belt 2, so that the inner frame 1 and the flexible belt 2 is stably fixed, discomfort of wearing or damage due to looseness is avoided, the structural strength is enhanced, and reliable connection of the internal components is ensured.

The technical features of the embodiments above may be combined arbitrarily. To make the description concise, not all possible combinations of the technical features in the above embodiments are described. However, as long as there are no contradictions in the combinations of these technical features, all of the combinations should be considered to be within the scope of the specification.

The embodiments above only represent several implementation modes of the present disclosure, and the description thereof is relatively specific and detailed, but it should not be construed as limiting the scope of the patent. It should be noted that for those skilled in the art, various modifications and improvements may be made without departing from the concept of the present disclosure, and all these modifications and improvements belong to the protection scope of the present disclosure. Therefore, the scope of protection of the patent application should be subject to the appended claims.

What is claimed is:

1. An intelligent wearable device, comprising:
an inner frame;
a flexible belt provided on an outer periphery of the inner frame;
an adjusting assembly circumferentially provided on the inner frame and located on a side of the flexible belt away from the inner frame, wherein the adjusting assembly abuts against the flexible belt; and
a driving assembly provided on an outer side of the adjusting assembly and connected to the adjusting assembly;
wherein the driving assembly is capable of rotating in a first direction, when the driving assembly rotates in the first direction, the adjusting assembly is driven to rotate in a first circumferential direction of the inner frame, so as to press the flexible belt to be deformed to reduce an inner diameter of the inner frame; or
the driving assembly is capable of rotating in a second direction, when the driving assembly rotates in the second direction, the adjusting assembly is driven to rotate in a second circumferential direction of the inner frame, the flexible belt is elastically reset to increase the inner diameter of the inner frame.

2. The intelligent wearable device according to claim 1, wherein the adjusting assembly comprises at least one rotating member in transmission connection with the driving assembly.

3. The intelligent wearable device according to claim 2, wherein the rotating member comprises a cam and a camshaft, the cam is longitudinally provided on the outer periphery of the inner frame and is located on the side of the flexible belt away from the inner frame, and the camshaft longitudinally extends through an end of the cam; a distance between a first portion of a side surface of the cam and the camshaft is a first radius, a distance between a second portion of the side surface of the cam and the camshaft is a second radius, and the first radius is larger than the second radius.

4. The intelligent wearable device according to claim 3, wherein the cam has a cylindrical structure, and outer peripheries of portions of the camshaft extending out of an upper end of the cam and a lower end of the cam are provided with tooth structures, respectively.

5. The intelligent wearable device according to claim 4, wherein a plurality of rotating members are provided, and a plurality of cams are evenly spaced apart on the outer periphery of the inner frame; and wherein a portion extending outward from a top portion of the inner frame and a portion extending outward from a bottom portion of the inner frame are respectively provided with a plurality of through holes that are evenly spaced apart, and upper end and lower end of each camshaft are respectively inserted in a pair of the through holes respective located at the top portion and bottom portion of the inner frame.

6. The intelligent wearable device according to claim 4, wherein the driving assembly is a middle frame having an annular structure, and an inner circumference of the middle frame is provided with inner tooth surfaces respectively engaged with outer tooth surfaces provided on an outer periphery of an upper end and an outer periphery of a lower end of the camshaft.

7. The intelligent wearable device according to claim 6, further comprising a damping member, wherein the damping member is provided at a bottom portion of the outer periphery of the inner frame, and a surface of the damping member is provided with a tooth surface engaged with the inner tooth surface.

8. The intelligent wearable device according to claim 7, wherein the damping member is a protrusion extending outward from an outer wall of the inner frame.

9. The intelligent wearable device according to claim 3, wherein the second portion of the side surface of the cam is opposite to the first portion of a side surface of the cam.

10. The intelligent wearable device according to claim 3, wherein the rotating member comprises an eccentric rotation shaft, a first radial portion, and a second radial portion, the first radial portion is a portion of the edge of the cam is farthest from the rotation shaft, the second radial portion is a portion of the edge of the cam is closest to the rotation shaft, the first radius is a distance from the first radial portion of the cam to the rotation shaft, and the second radius is a distance from the second radial portion of the cam to the rotation shaft.

11. The intelligent wearable device according to claim 3, wherein a surface of the cam is a curved surface with a smooth transition.

12. The intelligent wearable device according to claim 1, wherein the inner frame is flexible.

13. The intelligent wearable device according to claim 1, further comprising an upper cover and a lower cover, wherein a portion extending outward from a top portion of the inner frame and a portion extending outward from a bottom portion of the inner frame are respectively provided with a plurality of fixing holes that are evenly spaced apart, a side of the upper cover adjacent to the top portion of the inner frame and a side of the lower cover adjacent to the bottom portion of the inner frame are respectively provided with a plurality of fixing posts, the plurality of fixing posts of the upper cover are in one-to-one correspondence with the plurality of fixing posts of the lower cover, each fixing post of the upper cover and corresponding fixing post of the lower cover respectively extend through the corresponding fixing holes and are located at a same side of one of a plurality of bending positions of the flexible belt.

14. The intelligent wearable device according to claim 1, further comprising:
a plurality of detection sensors provided on a side of the flexible belt away from the adjusting assembly, the plurality of detection sensors being configured to monitor health condition of a user; and
a power supply provided on the outer peripheral of the inner frame and electrically connected to the flexible belt.

15. The intelligent wearable device according to claim 1, wherein the plurality of detection sensors comprise at least two of a heart rate sensor, a blood oxygen sensor and a temperature sensor.

16. The intelligent wearable device according to claim 1, wherein the first circumferential direction is the same as the first direction.

17. The intelligent wearable device according to claim 1, wherein the second circumferential direction is the same as the second direction and opposite to the first direction.

18. The intelligent wearable device according to claim 1, wherein the flexible belt is a closed loop structure.

19. The intelligent wearable device according to claim 1, wherein a contact portion between the flexible belt and the adjusting assembly is provided with a cushioning portion.

20. The intelligent wearable device according to claim 19, wherein the cushioning portion is a sponge or a foam.
